(19) United States
(12) Patent Application Publication
(10) Pub. No.: US 2004/0126356 A1
(43) Pub. Date: Jul. 1, 2004

(54) Compositions and methods for diagnosis and treatment of cardiovascular disorders

(76) Inventors: Gerald Pang, New South Wales (AU); Patricia Lynne Conway, New South Wales (AU); Robert Llewellyn Clancy, New South Wales (AU)

Correspondence Address: FISH & RICHARDSON PC, 225 FRANKLIN ST, BOSTON, MA, 02110, US

(21) Appl. No.: 10/399,932

(22) PCT Filed: Oct. 25, 2001
(86) PCT No.: PCT/IB01/02005

(30) Foreign Application Priority Data
Oct. 25, 2000 (AU) PR 1016

(51) Int. Cl. A61K 45/00; A61K 38/19

(52) U.S. Cl. 424/85.1

(57) ABSTRACT

There is disclosed a method of prophylactic or therapeutic treatment of a cardiovascular disorder comprising administering to a subject in need thereof an effective amount of one or more agents for upregulating a cytokine profile characteristic of a Th1 T-cell response relative to a cytosine profile of a Th2 T-cell response associated with the disorder. There is further disclosed compositions for use in the methods.

### TECHNICAL FIELD

The present invention relates to methods for diagnosis of cardiovascular disorders and to compositions suitable for use in therapeutic or prophylactic treatment of such disorders. In particular the present invention relates to methods and compositions suitable for the diagnosis and treatment of coronary artery disease.

### BACKGROUND OF THE INVENTION

Atheroma is the inflammatory process involving arteries that underpins coronary artery disease in particular and degenerative vascular disease in general. Data exists to support the concept that T lymphocytes drive inflammation within the atherosclerotic plaque. In particular, it has been reported that 2-10% of mononuclear cells in the plaque are T cells, two thirds of which are CD4+ve, and most of which express CD45RO, MHC class II, and IL-2R (L amon et el Immunology Today 18 (1997) 272-7). Pro-inflammatory cytokines such as IL-1, IL-6, TNF-α and INF-γ are secreted from cells within plaque, as are cell modifying factors such as PDGE, MCP-1, and M-CSF, and proteolytic enzymes such as matrix metalloproteinases, e.g. collagenase and gelatinase B (Lanmon et all, 1997).

The critical but complex relationship between T lymphocytes and macrophages within the plaque may be mediated in part by a receptor ligand couple through ligation of CD40L on activated plaque T cells by CD40 on macrophages (and other cells) to influence a range of outcomes including plaque remodeling, plaque rupture and antigen presentation (Lamon et al, 1997).

Recently, particular microbes have been linked to the promotion of atheroma The most characterised has been Chlamydia pneumoniae, though recent reviews have suggested that persistent infection in general may be linked to intimal inflammation and atheroma plaque growth (Saiku et a, Lancet 116 (1998) 983-5; Shar et al S Sfr Med J 82 (1992) 158-61; Mejer et al JAMA 281 (1999) 427). No data exists to clarify the basic mechanisms responsible for atheroma progression or processes whereby ‘epidemiologically-linked’ microbes facilitate atheroma growth.

There is a need for improved methods for assisting in the diagnosis of cardiovascular disorders which have basis in the immune response, e.g. coronary artery disease, and for compositions for the prophylaxis or therapy of such conditions.

### SUMMARY OF THE INVENTION

It is an aim of the present invention to overcome or ameliorate one or more of the problems of the prior art, or to at least provide a useful alternative.

The present invention is based on the identification of a major new mechanism for development of coronary artery disease, such as atheroma, due to the “Th2 cytokine” bias of modern living, not unlike the situation of allergy, also a disease of “modern living” linked to ‘Th2 bias’. Many factors modify the atheroma-promoting effect of Th2 inflammatory responses (e.g. lipid levels, smoking, hypertension, etc). Not wishing to be bound by any particular mechanism of action, the cause is probably an environmental effect on gut bacteria, replacing Th1 promoting microbes such as Lactobacilli with others linked with Th2 responses.

This new observation provides a unique opportunity for diagnostics and therapies to detect and modify respectively, atheroma-prone or high load atheroma subjects. In particular, reconstituting the gut with certain ‘traditional’ bacteria (probiotics) is identified as one useful therapeutic approach.

Diagnostics and therapy geared at additional specific microbes that further exacerbate the Th2 bias (eg C. pneumoniae and H. pylori) once established, are also specifically contemplated herein. The concept that ‘modern living atheroma’ is driven by altered cytokine patterns secondary to gut flora shifts, is consistent with the view that an essential difference between atheroma in developed versus developing countries, is the excess amount of inflammation in plaque in developed countries.

Thus, in broad terms the present invention is concerned with methods for diagnosing or detecting significant Th2-mediated atheroma, eg. coronary artery disease, based on the assessment of various markers and indicators of a Th2 response in blood (which interchanges with tissue spaces in the arterial wall), and with compositions capable of use as therapeutic or prophylactic agents able to promote a Th1 response and/or to suppress the Th2 response.

In particular, in one aspect of the present invention there is provided a method of prophylactic or therapeutic treatment of a cardiovascular disorder comprising administering to a subject in need thereof an effective amount of at least one agent for upregulating a cytokine profile characteristic of a Th1 T-cell response relative to a cytokine profile of a Th2 T-cell response associated with the disorder.

The upregulation of the cytokine profile characteristic of a Th1 T-cell response may be achieved by upregulating a Th1 T-cell response and/or suppressing Th2 T-cell response in the subject. Alternatively, the upregulating may be achieved by potentiating the activity of cytokines characteristic of a Th1 T-cell response and/or suppressing the activity of cytokines characteristic of a Th2 response.

A single agent or a plurality of agents may be administered to the subject to achieve the desired outcome. This may be obtained by administering an agent or agents which suppress the Th2 T cell response and thereby achieve a relative upregulation of the Th1 T cell response, or by administering an agent or agents which produce a measurable elevation in Th1 T cell response. Alternatively, one or more agents capable of measurably elevating the Th1 T cell response may be administered to the subject as well as one or more agents for suppressing the Th2 T cell response. Preferably, at least one agent capable of upregulating the Th1 T cell response and suppressing the Th2 T cell response will be administered

Typically, the method will comprise shifting the cytokine profile characteristic of a Th2 T-cell response to a cytokine profile characteristic of a Th1 T-cell response.

Accordingly, in another aspect of the present invention there is provided a method of prophylactic or therapeutic treatment of a cardiovascular disorder, comprising administering to a subject in need thereof an effective amount of at least one agent capable of upregulating a Th1 T-cell response, and/or at least one agent capable of suppressing a Th1 T-cell response associated with the disorder.

In yet another aspect of the present invention there is provided a method of prophylactic or therapeutic treatment of a cardiovascular disorder, comprising administering to a subject in need thereof an effective amount of at least one agent capable of suppressing the activity of cytokines characteristic of a Th2 T-cell response associated with the disorder, and/or at least one agent capable of potentiating the action of cytokines characteristic of a Th1 T-cell response.

In a further aspect of the present invention there is provided a method of altering cytokine balance in a subject with a cardiovascular disorder, comprising administering to a subject in need thereof of an effective amount of at least one agent capable of upregulating of a Th1 T-cell response, and/or at least one agent capable of suppressing a Th2 T-cell response associated with the disorder.

In still another aspect of the present invention there is provided a method of altering cytokine balance in a subject with a cardiovascular disorder, comprising administering to a subject in need thereof of an effective amount of at least one agent capable of suppressing the action of cytokines characteristic of a Th2 T-cell response associated with the disorder, and/or or at least one agent capable of potentiating the action of cytokines characteristic of a Th1 T-cell response.

Preferred agents for use in methods of the invention are microorganisms, or components, extracts or secreted products thereof capable of achieving the desired outcome. The microorganisms may for instance be yeasts, bacteria, and mixtures of these. Preferably, the microorganisms will be bacteria and more preferably, probiotic bacteria. Suitable probiotic bacteria may be selected from Lactobacillus spp. and/or Mycobacterium spp. Lactobacilli having the capability of suppressing the Th2 response and lower cholesterol are preferred. Particularly preferred are Lactobacillus acidophilus and Mycobacterium vaccae.

It will be understood that the microorganisms may be administered alive, inactivated or killed. Preferably, probiotic bacteria are administered as viable organisms.

However, the invention is not limited to the use of microorganisms and it will be understood that any agent capable of eliciting the upregulation of a cytokine profile characteristic of a Th1 T-cell response relative to that of a Th2 T-cell response may be utilised. Other agents include, for example, antibodies and binding fragments thereof. Anti-CD40 antibodies or binding fragments thereof are particularly preferred. In addition, other ligands for CD40 may be used.

The cytokine marker(s) may be any cytokine or cytokines characteristically associated with either a Th1 or a Th2 response. For example, for a Th1 response the cytokine may be interferon-γ or interleukin-12, while for a Th2 response the cytokines may be interleukin-4, interleukin-10, TGF-β and/or interleukin-13. However, it will be understood that any other cytokine marker is useful as long as it is a specific or identifiable marker for either a Th1 or Th2 response.

The treatments outlined above can be combined with the administration of one or more pharmaceutically active agents used to treat underlying conditions which may exacerbate the cardiovascular disorder, such as for example lipid-lowering drugs, anti-hypertensive agents and anti-diabetic agents.

The agent used to alter the T-cell response or to modulate the activity of the relevant cytokines can be administered prior to, simultaneously with or subsequent to one or more such pharmaceutically active agents.

The methods of the invention may also be effective in subjects in which the disturbance in cytokine balance or the lack of an appropriate T cell response is exacerbated by bacterial infection, bacterial antigens, polyclonal activators (e.g. endotoxin etc.), super antigens (e.g. from colonising bacteria) or autoantigens (within the plaque of blood vessel walls). Particularly relevant to the present invention is infection by, or bacterial antigen from, Chlamydia pneumoniae, Helicobacter pylori or non-typable Haemophilus influenzae.

Hence, in a still further aspect of the present invention there is provided a method of diagnosing or evaluating susceptibility to a cardiovascular disorder, comprising evaluating a T-cell response in a subject wherein an upregulated Th2 response and/or suppressed Th1 response is indicative of susceptibility to, or the presence of, the disorder.

In another aspect of the present invention there is provided a method of diagnosing or evaluating susceptibility to a cardiovascular disorder, comprising evaluating a T-cell response in a subject wherein suppressed activity or production of cytokines characteristic of a Th1 response and/or potentiated activity or production of cytokines characteristic of a Th2 response is indicative of susceptibility of the subject to, or the presence of, the disorder.

In a further aspect of the present invention there is provided a method of diagnosing a cardiovascular disorder or evaluating whether a subject is susceptible to the disorder, comprising:

(a) measuring one or more immunoglobulin levels affected by the disorder to obtain test data; and

(b) comparing the test data with reference data to evaluate whether the subject is susceptible to, or has, the cardiovascular disorder.

Preferably, the immunoglobulin is IgG and more preferably, the IgG2 subclass.

Preferably, the immunoglobulin is an antibody of the IgG2 subclass which is specific for pathogenic bacteria such as for example Chlamydia pneumoniae, Helicobacter pylori or non-typable Haemophilus influenzae. It will be clear to those skilled in the art that other specific antibodies may also be employed.

Preferably, a ratio of total IgG2 to IgG2 subclass specific antibody, or an altered ratio of total IgG2 subclass immunoglobulin to IgG2 subclass specific antibody will be used as an indicator of the presence of or susceptibility to the cardiovascular disorder.

The term ‘cardiovascular disorder’ is to be taken to encompass atheroma and degenerative vascular disease, and any cardiovascular condition or disease associated with inflammation of the coronary-arteries including 1 to 3 coronary artery disease.

Generally, the cardiovascular disorder will be a degenerative vascular disease and more usually, atheroma.

Specifically, methods of the invention have application for the treatment of subjects suffering from atheroma (as determined by angiography) with minimal or extensive coronary atherosclerosis but stable clinical disease, as well as atheroma subjects with unstable clinical disease associated with recent myocardial infarction or unstable angina.

Preferably, the T cell response will be evaluated by analysis of circulating T-cells. However, it will be understood that the T cell response may also be evaluated by measurement of any marker cytokine or cytokines characteristic of a particular T-cell response, such as for example, interferon-γ or IL-12 for a Th1 response or interleukin-4 and/or interleukin-13 for a Th2 response.

Compositions for use in the methods described herein are also specifically encompassed within the scope of the invention. Further, the use of the agents as described herein in the manufacture of a medicament or therapeutic composition for administering to a subject for the prophylaxis or therapeutic treatment of a cardiovascular disorder, is also specifically encompassed.

In addition, there are also provided kits for use in the methods of diagnosis or evaluation of the invention. A kit may for instance comprise one or more of reagents for performing the assays such as antibodies, buffers, controls and instructions for use.

The features and advantages of the present invention will be now be described hereinafter with reference to a number of preferred, non-limiting embodiments of the invention.

### BRIEF DESCRIPTION OF THE ACCOMPANYING DRAWINGS

FIG. 1 illustrates suppression of IL-4 secretion in whole blood by L. fermentum;

FIGS. 2A and 2B illustrate suppression of IL-4 secretion and potentiation of IFN-γ secretion by L. acidophilus, respectively;

FIGS. 3A and 3C illustrate secretion of IL-4 in C. pneumoniae seronegative and seropositive subjects with coronary vessel disease compared to normal subjects respectively;

FIGS. 4A and 4D illustrate secretion of IL-4 and IFN-γ in subjects with coronary vessel disease compared to normal subject respectively;

FIG. 5 illustrates the effect of Lactobacillus fermentum KLD on atherosclerosis in mice fed a high cholesterol diet; and

FIG. 6 illustrates inhibition of IL-4 production by treatment of whole blood cultures with anti-CD40 monoclonal antibody.

### DETAILED DESCRIPTION OF PREFERRED EMBODIMENTS OF THE PRESENT INVENTION

It has been observed that the presence of significant atheroma results in elevated blood levels of IL-4 and a concomitant reduction in IFN-γ levels. This alteration in the cytokine balance is indicative of a shift towards a Th2 response and is useful in the diagnosis of atheroma The observation also provides a sound basis for treatments which are aimed at altering the T cell response towards a Th1 response and thus, are beneficial in preventing and/or treating coronary artery disease and other cardiovascular disorders including atheroma which have basis in a similar underlying mechanism.

An example of possible therapeutic preparations contemplated herein are those which include probiotic bacteria (such as lactobacilli) which can drive the cytokine balance back towards a Th1 response and thus reduce progression of, prevent onset of or reverse the cardiovascular disorder. However, other agents and compositions, such as for example bacterial adjuvants as described further below that have the ability to shift the response from Th2 to Th1 are also useful in therapies for the conditions described herein.

Any method of detecting Th2 bias in circulating T cells, whether directly or indirectly such as by monitoring downstream effects of this bias such as IgG subclass variation or IgG subclass specific antibody variation as would occur in the production of antibody to C.pneumoniae or H.pylori (but not limited to those pathogens), would be useful as an indication of coronary artery disease. For example, IgG2 is relatively low when the cytokine patterns shift towards Th2. The thus altered ratio (or low levels) of total IgG2 subclass immunoglobulin or IgG2 subclass antibody specific for instance to C.pneumoniae or H.pylori, would indicate ‘atheroma-promoting’ cytokine bias.

Indeed, levels of immunoglobulins such as IgG2 subclass antibody may be measured and compared to reference levels or ratios to allow an evaluation to be made on whether a subject is susceptible to a cardiovascular disorder such as atheroma or otherwise has the disease. Suitable reference levels or ratios will generally be based on corresponding measurements obtained from healthy individuals and will typically comprise mean values derived from a representative cohort of the population in accordance with conventional methodology.

Further, methods of preventing, treating or reversing atheroma contemplated by the present invention include any treatment that shifts or otherwise alters the cytokine balance towards a Th1 response, such as the administration of probiotic bacteria (especially Lactobacilli species). For instance, Lactobillus acidophilus can downregulate IL-4 and upregulate IF-γ secretion from T cells within the spleen (i.e. circulating cells) and thus have application to the treatment of atheroma and other such cardiovascular disorders. Other treatments include the administration of any factor that suppresses Th2 cytokine secretion or inhibits action of these cytokines, and/or any treatment that promotes secretion or activity of Th1 cytokines such as INF-γ.

It will also be clear to those skilled in the art that any treatment that specifically modifies the level or pattern of cytokine secretion from circulating T cells specifically reactive to antigens (eg C.pneumoniae or H.pylori) or non-specific activating factors (eg polyclonal activators, endotoxin or superantigens) can be employed as is contemplated herein.

Further, treatments combining probiotics or other agents capable of altering the cytokine balance towards a Th1 response with any existing therapy aimed at ‘risk factors’ ) eg. lipid-lowering drugs, anti-hypertensive agents and the like may also be usefully employed. Many additional factors drive atheroma (eg blood lipids, diabetes, hypertension, smoking) and the combination of therapies which alter cytokine balance with those which treat the underlying condition are also contemplated herein.

Typically, a sample will be obtained from the subject for evaluating T-cell cytokine profile and/or the T-cell response. The sample may be a whole blood sample, a cellular component of whole blood, isolated cells or for instance a tissue biopsy sample suitable for assaying.

The microorganisms may be selected from bacteria and yeast strains including saccharomyces spp. such as Saccharomyces cerevisae and Saccharomyces boulardii. Preferably, the bacteria will be a probiotic bacteria. Alternatively, components, sonicates, extracts or secreted products, or mixtures thereof of the microorganism(s) may be used. Extracts include, for example, cell wall fractions. Components of the microorganism(s) may comprise antigens for instance, antigenic peptides and the like obtained by enzymatic treatments well within the scope of the skilled addressee.

Bacteria may, for example be selected from, but not limited to, Lactobacillus species, lactic acid bacteria, Mycobacterium species and Bifidobacterium species. Even more preferred is the use of Lactobacillus acidophihis (L. acidophilus), Lactobacillus fermentum (L. fermentum) or Mycobacterium vaccae (M. vaccae), or components extracts, sonicates, secreted products or mixtures thereof that are capable of inducing a Th1 cellular response. Specially preferred is L. acidophilus, L. fermentum or M. vaccae which may be used live or as an inactivated preparation, as long as they are capable of inducing the desired Th1 T-cell response.

Preferably, L. acidophilus and L. fermentum is used as a live preparation. Other bacteria may also be used (whether they have probiotic effect or not), for example the well known adjuvating bacteria such as for example L. casei, L. plantarum, L. rhamnosus, Bifidobacterium breve and the like.

The dosage of the microorganism or extracts and the like thereof administered to the subject may vary according to the nature and severity of the cardiovascular disorder, whether the agent is administered for prophylactic or therapeutic purposes and the type of organism involved. The treatment parameters as well as the required dosage can be readily determined by the person skilled in the art.

Preferably, a microorganism or microorganism-containing composition will be in tablet or capsule form. However, it will be clear to those skilled in the art that the microorganism may be provided in a liquid or other form of solid preparations. In particular, the microorganism may also be provided as a food source such as a yoghurt or other dairy product, or similar non-dairy products based for example on soy.

The microorganisms or the like will generally be administered orally at regular intervals, and typically daily for the duration of the treatment period which may extend for a period of up to several months or more. Preferably, the microorganisms will be administered in a dosage of log 3 to log 12 per day. The dosage of probiotic bacterium when administered as live whole bacterium may be in the range of from about 1×10⁸ to about 1×10¹² organisms.

However, other agents capable of upregulating a cytokine profile characteristic of a Th1 T-cell response in accordance with methods of the invention may also be utilised. The skilled addressee will be able to readily identify such other agents by routine trial and experimentation on the basis of the teachings provided herein. Such other agents may include, for instance antibodies and binding fragments thereof. In this regard, the preset inventors have found that levels of blood T-cell secreted IL-4 associated with atheroma correlates with the extent of the coronary artery disease. This impressive correlation fits well with observations by the present inventors that T-cell mediated inflammation is driven by ligation of CD40L on CD4+ T-cells by CD40 on a range of structural and circulating cells including platelets. In particular, platelets appear to be an important factor for the production of IL-4 as a result of ligation of CD40L expression on activated CD4+ T-cells by CD40 expressed on the platelets.

Accordingly, administration of an agent capable of inhibiting ligation of CD40L with CD40 such as an antibody, and particularly an anti-CD40 antibody or binding fragments thereof, may alter the cytokine profile characteristic of a Th2 response in the patient. By binding fragments is meant fragments of an antibody which retain the binding capability of the antibody and include Fab and (Fab′)₂ fragments as may be obtained by papain or pepsin proteolytic cleavage, respectively. In addition, other ligands for CD40 as will be known the skilled addressee or peptide fragments thereof may be administered for achieving the desired upregulation of a Th1 T cell response relative to a Th2 T cell response. Appropriate such ligands and agents can be readily identified utilising the methodology as disclosed in the accompanying Examples. Such agents may be administered intravenously, intramuscularly, or subcutaneously, or by any other route deemed appropriate.

Such agents and other agents like microorganism extracts, sonicates and the like may be formulated into pharmaceutical compositions incorporating pharmaceutically acceptable carriers, diluents and/or excipients for administration to the intended subject. The dosage of such other active agents will typically be in accordance with conventional treatment regimens for their use taking into account such factors as age, weight, nature of the condition being treated and the general health of the subject as will be readily appreciated.

Pharmaceutical forms include aqueous solutions suitable for injection, and powders for the extemporaneous preparation of injectable solutions. Such injectable compositions will be fluid to the extent that syringability exists and typically, will be stable to allow for storage after manufacture. The carrier may be a solvent or dispersion medium containing one or more of ethanol, polyol (eg glycerol, propylene glycol, liquid polyethylene glycol and the like), vegetable oils, and suitable mixtures thereof. Fluidity may be maintained by the use of a coating such as lecithin, by the maintenance of the required particle size in the case of a dispersion and by the use of surfactants.

Injectable solutions will typically be prepared by incorporating the active agents in the desired amount in the appropriate solvent with various other components enumerated above. Generally, dispersions will be prepared by incorporating the active agents into a vehicle which contains the dispersion medium and other components. In the case of powders for the preparation of injectable solutions, preferred methods of preparation are vacuum drying and freeze-drying techniques which yield a powder of the active agent.

For oral administration, agents may be formulated into any orally acceptable carrier deemed suitable. In particular, the active ingredient may be formulated with an inert diluent, an assimilable edible carrier or it may be enclosed in a hard or soft shell gelatin capsule. Alternatively, it may be incorporated directly into food as indicated above. Moreover, an active agent may be used in the form of ingestable tablets, troches, capsules, elixirs, suspensions, syrups, and the like.

A composition of the invention may also incorporate one or more suitable preservatives such as sorbic acid. In many cases, a composition may furthermore include isotonic agents such as sugars or sodium chloride.

Tablets, troches, pills, capsules and the like may also contain one or more of the following: a binder such as gum tragacanth, acacia, corn starch or gelatin; excipients such as dicalcium phosphate; a disintegrating agent such as corn starch, potato starch, alginic acid and the like; a lubricant such as magnesium sterate; a sweetening agent such as sucrose, lactose or saccharin or a flavouring agent. When the dosage unit form is a capsule, it may contain in addition to one or more of the above ingredients a liquid carrier. Various other ingredients may be present as coatings or to otherwise modify the physical form of the dosage unit. For instance, tablets, pills or capsules may be coated with shellac, sugars or both. In addition, an active agent may be incorporated into any suitable sustained-release preparation or formulation.

Pharmaceutically acceptable carriers, diluents and/or excipients include any suitable conventionally known solvents, dispersion media and isotonic preparations or solutions. Use of such ingredients and media for pharmaceutically active substances is well known. Except insofar as any conventional media or agent is incompatible with the active agent, use thereof in therapeutic and prophylactic compositions is contemplated. Supplementary active ingredients can also be incorporated into the compositions if desired.

As will be appreciated, the amount of agent or agents in such compositions will be such that a suitable effective dosage will be delivered to the subject taking into account the proposed mode of administration.

Dosage unit form as used herein is to be taken to mean physically discrete units suited as unitary dosages for the subject to be treated, each unit containing a predetermined quantity of active agent calculated to produce the desired therapeutic or prophylactic effect in association with the relevant carrier, diluent and/or excipient.

The agent may be administered in conjunction with one or more antibiotics or one or more other pharmaceutically active agents for treating the cardiovascular disorder or any underlying condition that exacerbates the disorder, and may be administered prior to, simultaneously with or subsequent to antibiotic therapy or therapy with other active agents.

### EXAMPLES

### Example 1

### Lactobacillus Inhibits IL-4 Secretion

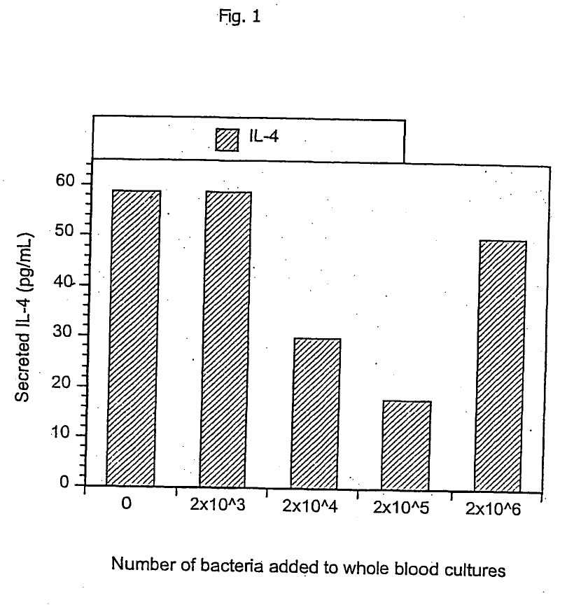

To determine whether Lactobacillus has the capacity to regulate IL-4 production, graded doses of Lactobacillus fermentum (strain VRI 002 available from the Culture Collection of the School of Microbiology and Immunology at the University of New South Wales, Sydney, Australia) were added to cultures containing equal volumes of heparinized whole blood from a normal healthy subject and AIM-V serum free medium. Control cultures contained medium alone. All cultures were stimulated with Con A (5 ug/ml). After incubation for 24 hrs, the amount of secreted IL-4 was determined by capture IL-4 ELISA. As shown in FIG. 1, IL-4 secretion was inhibited in a dose dependent manner in the presence of Lactobacillus fermentum with maximal effect occurring at 2×10⁵ bacteria per culture. This data indicates that Lactobacillus fermentum is effective in down-regulating IL-4 mediated inflammation associated with a Th2 response.

### Example 2

### Effect of Probiotic Bacteria on Th1/Th2 Cytokine Response

To determine whether probiotic can down-regulate a Th2 and up-regulate a Th1 cytokine response, C57/B16 mice were fed intragastrically, various numbers of Lactobacillus acidophilus (strain VRI 001 available from the Culutre Collection of the School of Microbiology and Immunology, University of New South Wales, Sydney, Australia) using a feeding needle on consecutive days for 2 weeks, after which they were sensitised with 8 μg of ovalbumin (OVA) and aluminium hydroxide in 0.2 mL phosphate-buffered saline administered by peritoneal injection. The mice were further fed ten times with L. acidophilus every two days for two weeks before they were sacrificed. Lymphocytes were isolated by teasing spleens through a sieve, washed with PBS, and resuspended at 10×10⁶ cells/ml culture medium.

One mL aliquots of the cell suspension were then dispensed into wells of a 24-well flat-bottomed microtitre plate and stimulated with OVA (5 μg/mL). After incubation for 4 days the supernatants were collected and assayed for L-4 and IFN-γ production by standard ELISA techniques using IL-4 or IFN-γ monoclonal antibody pairs.

Briefly, wells of a 24-well microtitre plate were coated with a capture anti-IL-4 antibody. After incubation at room temperature for 1 hr, the wells were washed and biotinylated anti-IL4 antibody was added to each well. Following incubation for a further 1 hr, the wells were washed and strepavidin-peroxidase conjugate was added to each well. After incubation for 30 mins, the wells were washed and then TMB substrate was added. The colour development was read at 450/620 nm in an ELISA plate reader. The level of IL-4 in unknown samples was quantitated by interpolation using a standard curve. A similar procedure was used for measurement of IFN-γ.

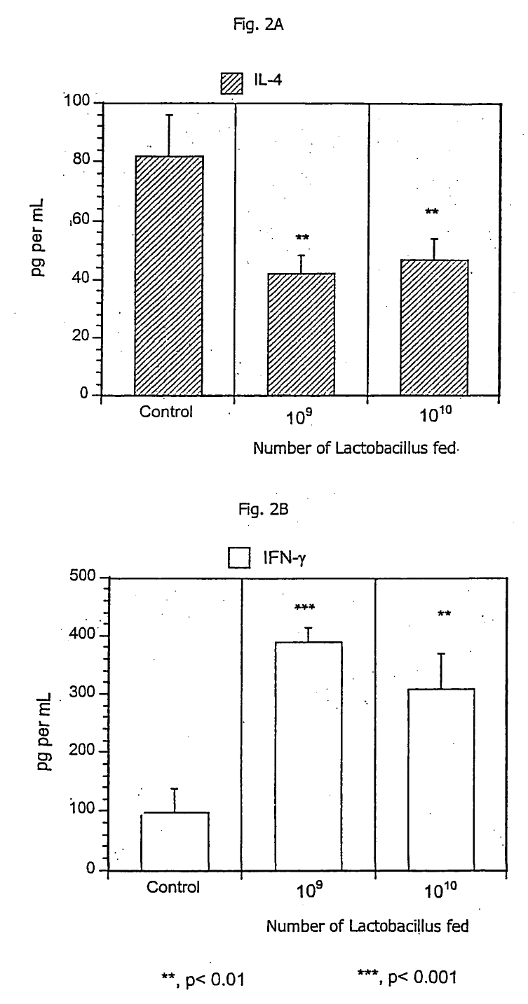

The results are shown in FIG. 2A and FIG. 2B. As can be seen, FIG. 2A demonstrates that feeding L. acidophilus resulted in the suppression of IL-4 production in a dose-dependent manner whereas FIG. 2B shows that production of IFN-γ was enhanced. Accordingly, increased production of secreted IL-4 in whole blood correlates with severity of disease in subjects with coronary artery disease.

### Example 3

### Subject Selection and Measurements

3.1 Subjects. Subjects presenting at the John Hunter Hospital (Newcastle, Australia) were selected following angiographic study. Risk factors were recorded (ipid profile, hypertension, diabetes, smoking, family history). The following groups were identified: (a) minimal coronary atherosclerosis (n=100); (b) extensive coronary atherosclerosis (>50% three major vessel involvement) with stable clinical disease (n=100), and (c) extensive coronary atherosclerosis—unstable clinical disease (n=100) (recent myocardial infarction or unstable angina).

Blood (20 ml) was taken following angiography from the selected subjects for antibody and T cell studies. The number of angiographic studies at the John Hunter Hospital (Newcastle, Australia) is about 30-40/week, with the distribution being approximately 10-15% with normal arteries or minimal disease and 20-30% with triple artery disease, of which about one third has unstable clinical disease and two thirds have stable clinical disease.

3.2 Anti-Clamydia Pneumoniae Antibody. The antibody was detected by a micro-immunofluorescence test for immunoglobulin IgG to C.pn-specific antigen (Chlamydia-cel Pn kit, CeLLabs Pty Ltd, Australia). IgG subclass antibody was detected using specific IgG subclass antisera.

3.3 T-cell proliferation. Whole blood lymphocyte culture was performed in triplicate in 96-well round-bottomed microtitre plates. Heparinised blood was diluted 1:1 (v/v) with AIM-V serum free-medium containing graded amounts (0.1, 1.0, 10 μg/ml) of Chlamydia pn elemental bodies (EB) prepared as described below. All subjects were stimulated in addition with C. trachomatis or EB antigen (0.1, 1.0, 10 μg/ml) as an ‘irrelevant’ antigen control. After five days at 37° C. in 5% CO₂, titrated thymidine (0.5 μCi per culture) was added for the final six hours before harvesting and counting.

3.4 Cytokine production. Cytokine-based whole blood assays for detection of EB-reactive T cells were used. Heparinised blood was diluted 1:1 (v/v) with AIM-V medium with or without various concentrations of EB antigens in wells of a 96-well round-bottomed microtitre plate. For measuring the production of IL-4, some wells were pre-coated with a capture monoclonal anti-IL4 antibody (Endogen, CSL). The cultures were incubated at 37° C. in a 5% CO₂ atmosphere for 2448 hours after which time the plasma supernatants were collected for IL-2, IL-10 and IFN-γ assays (Endogen kits, CSL). Captured IL-4 together with appropriate standards were directly determined in the wells following washing and the addition of developing anti-IL-4 antibody as described in the assay kit. The whole blood assay for measuring antigen-reactive T cells and cytokine production profiles had been validated for studies in human subjects with H.pylori infection.

3.5 Preparation of elemental bodies from: Chlamydia pn. A HeLa cell 229 adapted C.pn Kajaani strain obtained from Professor P Saikku (University of Helsinki, Finland) was grown in HeLa cells in culture flasks containing RPMI 1640 medium supplemented with 5% foetal calf serum (FCS) and streptomycin at 37° C. in a 5% CO₂ humidified atmosphere. Chlamydia elemental bodies were isolated from cultured cells after three days. The cells were detached from the flask using a sterile scraper, washed and suspended in phosphate buffered saline (PBS) and the inclusion bodies disrupted by sonication. After removal of cell debris by centrifugaton, the EB material was collected by ultracentrifugation at 30,000 g. The EB material was then resuspended in PBS and layered onto a 30-60% Nycodenz solution (Nycomed, Norway). After centrifugation, the EB materials collected above the 60% gradient were washed and then inactivated with 1% formaldehyde for 24 hours. After extensive washing, the EB material was resuspended in PBS and the protein concentration determined (Pierce Protein Kit). EB antigens obtained from Professor Saikku and colleagues were also used in the study for comparison. A similar method was used for an elemental body antigen preparation from C. trachomatis (with samples again being provided by Professor P Saikkdu).

3.6 Specific cloned proteins. Cloned antigens from C.pn supplied by Drs. Saikku and Makela (Finland—above) were tested for cytokine balance (above). The cloned antigens comprised MOMP, OMP2 and HSP60 as recombinant proteins produced in B.subtilis. These were tested at 1 μg/ml.

In particular, heparinised whole blood was collected from patients with coronary atherosclerosis who were either seropositive (n=117) or seronegative (n—27) for C pneumoniae. After incubation overnight at 37° C. as above, secreted IL-4 was measured by capture ELISA while IFN-γ was measured in plasma supernatant.

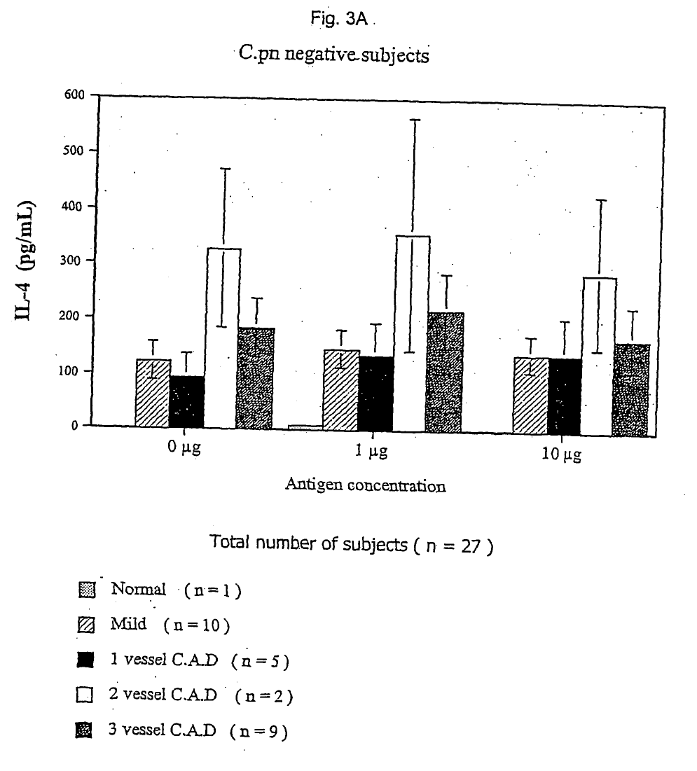

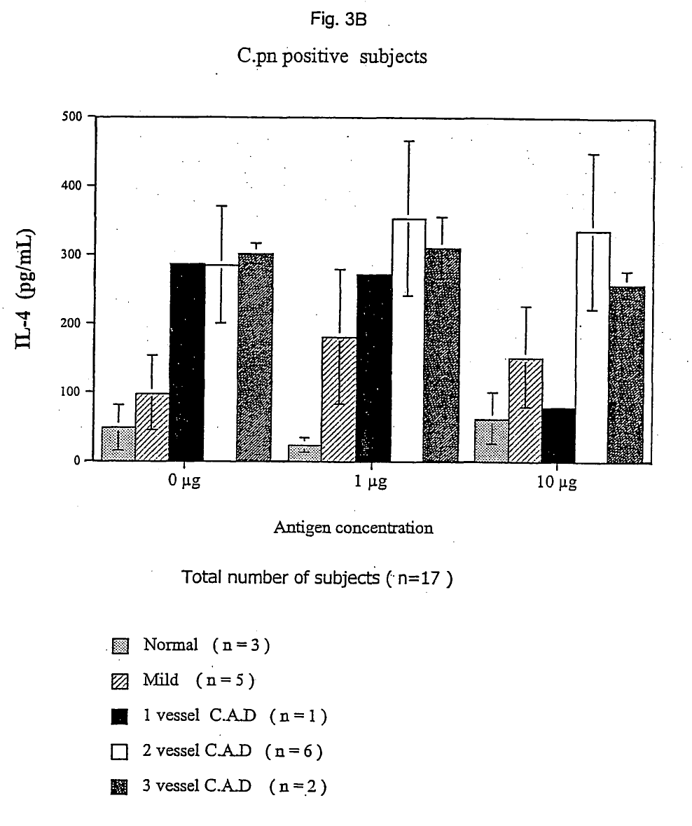

As shown in FIGS. 3A and 3B, higher levels of IL-4 were detected in subjects with 2-3 coronary vessel disease compared to subjects With mild or 1 vessel disease. Low to undetectable levels were observed in normal subjects. In C pneumoniae seropositive subjects, higher levels of secreted IL-4 were detected in those with 1-3 vessel disease compared to seronegative subjects especially those with 1 vessel disease, suggesting that increased production of secreted IL-4 is associated with infection status. However, in all subjects studied, IL-4 secretion was not dependent on stimulation with C pneumoniae antigens in culture, indicating that spontaneous production of IL-4 was a result of activated T-cells in vivo which are no longer responsive to further antigen stimulation in culture. When the data from the 44 subjects were combined the results were similar in that irrespective of antigen stimulation the levels of secreted ILL in whole blood cultures correlated with the extend of disease.

### Example 4

### Pattern of Spontaneous T Lymphocyte Activation

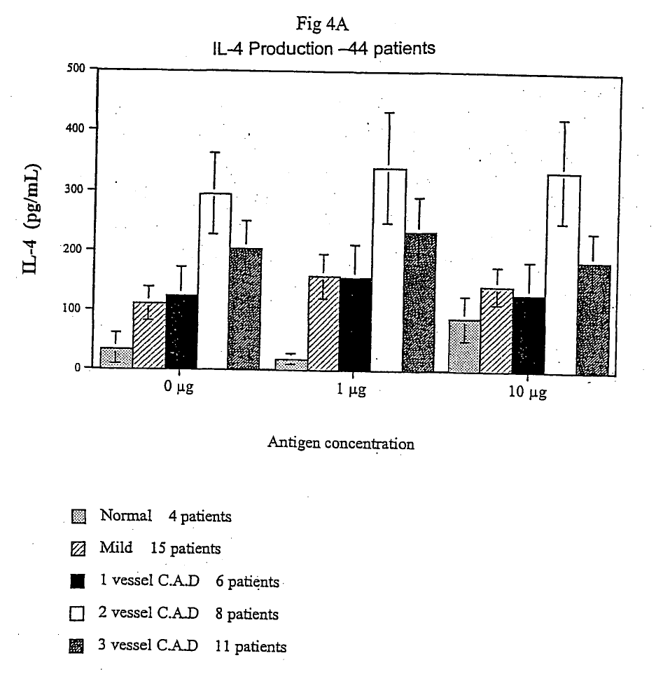

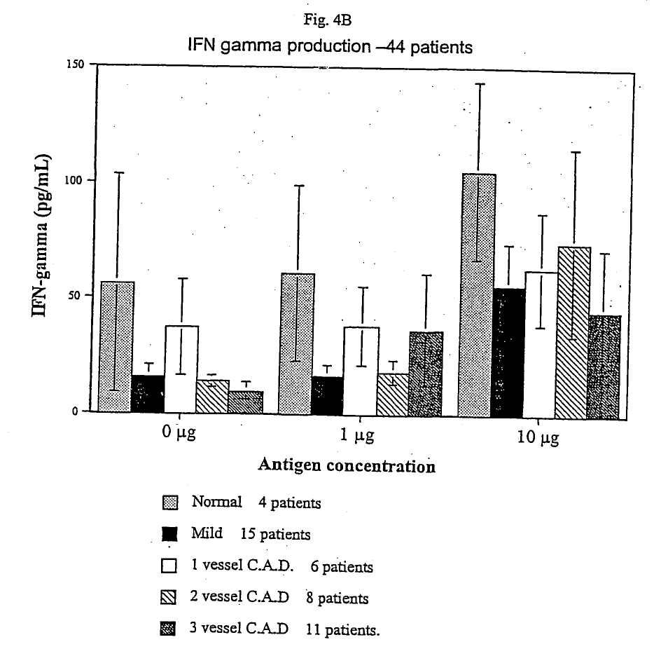

In marked contrast, there was inverse relationship between secreted IL-4 and IFN-γ production (see FIGS. 4A and 4B). However, there was no correlation between levels of IFN-γ and the severity of disease indicating the inflammatory response in atheroma is driven by CD4+ Th2 helper cell-mediated inflammation with upregulation of IL-4.

In particular, the results of spontaneous cytokine production show a significant difference between those with ‘normal’ coronary angiograms and those with two or three vessel disease (representing ‘high load’ atheroma), with those defined as mild or minimal coronary atherosclerosis being intermediate in amount of IL-4 produced. With respect to INF-γ, a difference between normal and ‘atheroma-detected’ subjects was found to be present, with the ‘normal’ subjects having higher levels. Differences between mild and severe atheroma for INF-γ is less marked than is the level of difference seen with L-4. Talcen together, these results clearly show that there is a shift in the Th1-Th2 balance correlating with the amount of atheroma.

It is concluded that subjects with a ‘set’ towards responding to stimuli of T cells with a Th2 pattern cytokine response, promote excessive accumulation of atheroma in blood vessel walls, as a result of the pathways of the inflammatory response linked to Th2 T cell activation. As cytokines measured here are spontaneously secreted from T cells in whole blood culture, activation has occurred in-vivo. Stimuli could include polyclonal activators (e.g. endotoxin from gut flora), super antigens (e.g. from colonising bacteria), autoantigens (including antigens within the plaque or blood vessel wall) or specific antigens, especially from microbes in a colonising or parasitic relationship with the host (e.g. Chlamydia pneumoniae, Helicobacter pylori, non-typable H. influenzae etc). The latter is consistent with the view that “chronic infection unrelated to particular microbial species” is a ‘risk factor’ for atherosclerosis progression rather than C. pneumoniae having an unique antigenic role (Groyston J T, Kuo Coulson A S et al, Circulation (1995) 92:3397-3400; Bachmaier K, Neu N et al, Science (1999) 283:1335-1339; Mejer D, Derby L E et al, JAMA (1999) 18:272-277). In addition, the data in FIGS. 3A and 3B show a trend towards greater ‘Th2-polarisation’ in cultures stimulated with C.pneumoniae antigen, consistent with the notion that within the context of a ‘Th2 set’ of the immune system, particular microbes may enhance the drive towards a Th2 response and thus further progress the atheroma plaque. Circulating cells would interchange with those included in atheroma plaque. Thus, chronic infection can exacerbate the Th2 bias in subjects with significant atheroma However, the present data on subjects with and without Chlamydial infection show that the basic “set” of Th2 cytokines is independent of Chlamydial infection (although the infection may exacerbate the bias as mentioned above).

This study supports the conclusion that the pattern of spontaneous T lymphocyte activation correlates with the amount of atheroma generally, but in particular in the coronary arteries.

### Example 5

### Effect of Feeding Lactobacillus on Atherosclerosis in Mice Fed a High Cholesterol Diet

The effect of a high cholesterol diet on the development of atherosclerosis as assessed by the formation of fatty streak in the aortic sinus (root) of mice was determined.

The diet contained the following ingredients:

| | g/100 g |
|---|---|
| Sucrose | 51.3 |
| Casein (acid) | 20.0 |
| Canola oil | 1.00 |
| Cocoa butter | 15.00 |
| Cellulose | 5.10 |
| DL-methionine | 0.30 |
| AIN93G minerals | 3.50 |
| AIN93G Vitamins | 1.00 |
| Choline Chloride 50% w/w | 1.00 |
| Sodium Cholate | 0.50 |
| Cholesterol | 1.00 |
| DL α-Tocopherol acetate | 0.26 |

Briefly, C57/B16 male mice (3 weeks old) were placed on a high cholesterol diet (HCD) or a cholesterol free normal diet, and with free access to drinking water. Groups of mice (n=10) were fed HCD for one week and then placed on a feeding regimen comprising Lactobacillus fermentum (VRI 002). The dose was administered orogastrically 3 times per week with a 200 μl sample of a washed bacterial suspension from an overnight culture resuspended to give a final density of between log 9.5 and log 10.5 organisms. Control mice were dosed with 200 μl of saline alone. After 5 weeks, two groups of mice were immunised subcutaneously with 0.1 mL of 5 mg/mL killed Mycobacterium tuberclosis (MT, Difco) emulsified in incomplete Freund's adjuvant. The rationale for the immunisation step was based on a recent report which suggests that activation of the immune system by immunisation with killed bacteria can lead to the acceleration of fatty streak formation in the aorta sinus (George J et al. Ateriosclerosis, Thrombosis and Vascular Biology, 1999, 19: 505-510).

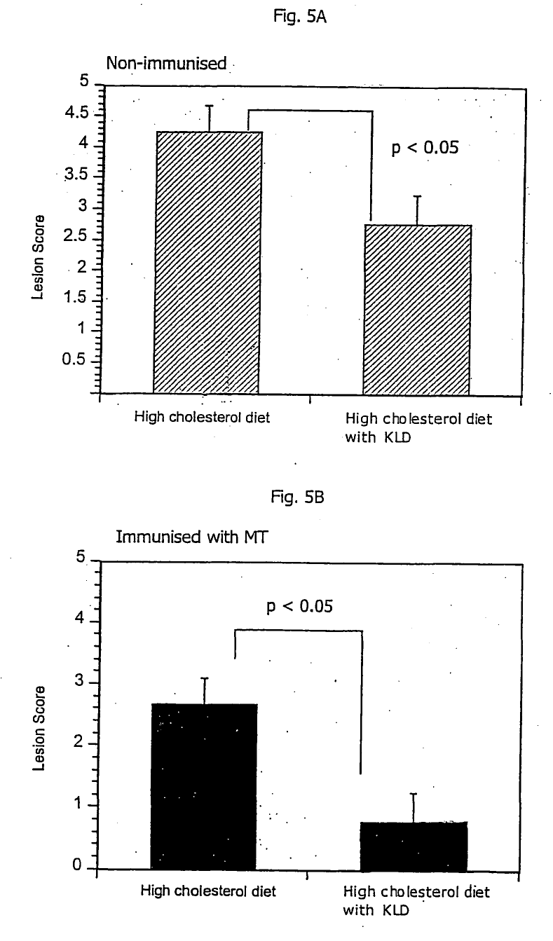

All mice were sacrificed at 7 weeks after commencement of the HCD and probiotic treatment. Blood was collected by cardiac puncture. The heart was removed en bloc and the upper section containing the aortic sinus (root) was excised and fixed in 10% formalin in PBS. After fixing overnight in formalin/PBS, the tissue was embedded n OCT medium and frozen before sectioning in a cryostat. Six to seven sections (8-10 μm thick) were taken and stained with oil Red O. Lesion areas per section were scored by a blind observer. A 0-5 lesion scoring system was adopted according to the presence of fatty streak formation. As shown in FIG. 5A, mice fed HCD alone had more formation of fatty streak than those treated with Lactobacillus. Similar results were obtained with mice immunised with MT (see FIG. 5B) although in these mice the amount of lesion was lower than non-immunised groups, suggesting that immunisation may limit atherogenesis.

### Example 6

### IL-4 Production in Whole Blood Cultures from Patients with Coronary Artery Disease is Inhibited by Anti-CD40 Monoclonal Antibody

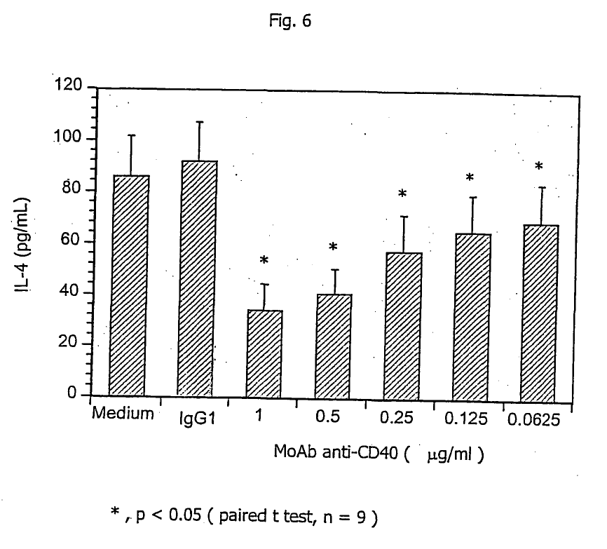

Heparinised blood was collected from subjects with coronary artery disease and cultured in equal volume of serum-free AIM-V medium (300 μL total volume) containing graded concentrations of anti-CD40L antibody in a 96-well flat-bottomed coated with anti-IL-4 antibody. Control cultures contained medium alone or a mouse IgG1 isotype control. After incubation for 24 hrs, the amount of L-4 secreted was measured by a capture ELISA assay. As shown in FIG. 6, IL-4 production was inhibited by anti-CD40 in a dose-dependent manner compared with control (p<0.05 for 9 subjects) while the addition of mouse IgG1 isotype control or anti-CD40L (data not shown) had no effect. The result showed that the engagement of CD40 is critical for the production of IL-4 whole blood culture.

Although the present invention has been described with reference to preferred embodiments, the skilled addressee will understand that numerous variations and modifications are possible without departing from the scope of the instant invention.

1. A method of upregulating a cytokine profile characteristic of a Th1 T-cell response relative to a cytokine profile of a Th-2 T-cell response associated with inflammation of blood vessels in a cardiovascular disorder, comprising administering to a subject in need thereof an effective amount of one or more probiotic agents for prophylaxis or treatment of the inflammation.

2. A method according to claim 1 wherein the method is a method of treating the inflammation.

3. A method according to claim 1 comprising shifting the cytokine profile characteristic of a Th2 T-cell response to a cytokine profile characteristic of a Th1 response.

4. A method according to claim 1 comprising administering a probiotic agent capable of upregulating a Th1 T-cell response and suppressing a Th2 T-cell response in the subject.

5. A method according to claim 1 comprising administering a probiotic agent capable of potentiating the action of cytokines characteristic of a Th1 T-cell response and suppressing the action of cytokines characteristic of a Th2 response in the subject.

6. A method according to claim 1 comprising administering a probiotic agent capable of upregulating a Th1 T-cell response in the subject.

7. A method according to claim 1 comprising administering a probiotic agent capable of potentiating the action of cytokines characteristic of a Th1 T-cell response in the subject.

8. A method according to claim 1 comprising administering a probiotic agent capable of suppressing a Th2 T-cell response in the subject.

9. A method according to claim 1 comprising administering a probiotic agent capable of suppressing the action of cytokines characteristic of a Th2 T-cell response in the subject.

10. A method according to claim 1 wherein the one or more probiotic agents comprises a microorganism, extract or sonicate, or a mixture of some or all of the foregoing.

11. A method according to claim 10 wherein the extract comprises a cell wall fraction of the microorganism.

12. A method according to claim 11 wherein the microorganism is selected from the group consisting of yeast and bacteria.

13. A method according to claim 12 wherein the microorganism is a probiotic bacterium.

14. A method according to claim 13 wherein the probiotic bacterium is selected from the group consisting of Lactobacillus and Mycobacterium species.

15. A method according to claim 14 wherein the Lactobacillus species is capable of suppressing a Th2 response and lowering cholesterol level in the subject.

16. A method according to claim 13 wherein the probiotic bacterium is selected from Lactobacillus acidophilus, Lactobacillus fermentum, and Mycobacterium vaccae.

17. A method according to claim 12 wherein the microorganism is a bacterium selected from the group consisting of Lactobacillus casei, Lactobacillus plantarum, Lactobacillus chamnosus and Bifidobacterium breve.

18. A method according to claim 10 wherein the microorganism is viable.

19. A method according to claim 1 or 2 further comprising administering to the subject an effective amount of at least one pharmaceutically active agent for treating the subject in addition to the probiotic agent for up regulating a cytokine profile characteristic of a Th1 T-cell response.

20. A method according- to claim 19 wherein the pharmaceutically active agent is selected from the group consisting of lipid-lowering drugs, anti-hypertensive agents and anti-diabetic agents.

21. A method according to claim 19 wherein the probiotic agent for up regulating the cytokine profile characteristic of the Th1 T-cell response is administered to the subject prior to, simultaneously with or subsequent to at least one pharmaceutically active agent.

22. A method according to claim 1 wherein the Th2 T-cell response associated with the disorder is exacerbated by bacterial infection, bacterial antigens, polyclonal activators, superantigens or autoantigens.

23. A method according to claim 22 wherein the infection is by, or the bacterial antigen is from, Chlamydia pneumoniae, Helicobacter pylori or non-typable Haemophilus influenzae.

24. A method according to claim 1 or 2 wherein the cardiovascular disorder is selected from stable or unstable clinical cardiovascular disease, degenerative vascular disease, atheroma and coronary artery disease.

25. A method according to claim 2-4 wherein the cardiovascular disorder is selected from the group consisting of subjects suffering from atheroma with stable or unstable clinical disease.

26. A method of diagnosing or evaluating susceptibility to inflammation of blood vessels associated with a cardiovascular disorder, comprising evaluating a T-cell response in a subject wherein an upregulated Th2 T-cell response and/or suppressed Th1 T-cell response is indicative of susceptibility to, or the presence of, the disorder.

27. A method according to claim 26 comprising determining whether the subject has an upregulated Th2 T-cell response and a suppressed Th1 T-cell response.

28. A method according to claim 26 wherein the evaluating comprises determining whether the activity or production of one or more cytokines characteristic of the Th1 T-cell response is suppressed and/or whether the activity or production of one or more cytokines characteristic of a Th2 T-cell response is potentiated.

29. A method according to claim 28 wherein the evaluating comprises determining whether the activity or production of one or more cytokines characteristic of Th1 T-cell response is suppressed and whether the activity or production or one or more cytokines characteristic of a Th2 T-cells response is potentiated.

30. A method according to claim 28 or 29 wherein the cytokine or cytokines are selected from the group consisting of IFN-γ, IL-4, IL-10 and IL-12.

31. A method according to any one of claims 26 to 30 wherein the T-cell response is evaluated by analysis of circulating T-cells.

32. A method of diagnosing a cardiovascular disorder associated with inflammation of blood vessels or evaluating whether a subject is susceptible to the inflammation, comprising:
(a) measuring one or more immunoglobulin levels affected by the disorder to obtain test data; and
(b) comparing the test data with reference data to evaluate whether the subject is susceptible to, or has, the inflammation,
wherein the one or more immunoglobulin levels are selected from the group consisting of total immunoglobulin isotype levels and levels of total immunoglobulin isotype subclasses.

33. A method according to claim 32 comprising measuring one or more IgG levels.

34. A method according to claim 33 comprising measuring total IgG2 subclass immunoglobulin.

35. A method according to claim 33 comprising measuring the level of an IgG2 subclass specific antibody.

36. A method according to claim 33 wherein a ratio of total IgG2 subclass to IgG2 subclass specific antibody, or an altered ratio of total IgG2 subclass to IgG2 subclass specific antibody, is indicative of susceptibility to, or presence of the disorder.

37. A method according to claim 32 wherein the cardiovascular disorder is selected from subjects suffering from stable or unstable clinical cardiovascular disease, degenerative vascular disease, coronary artery disease and atheroma.

38. A kit when used in a method of diagnosing a cardiovascular disorder or evaluating whether a subject is susceptible to the disorder, wherein the method involves measuring one or more immunoglobulin levels effected by the disorder to obtain test data, and comparing the test data with reference data to evaluate whether the subject is susceptible to, or has, the cardiovascular disorder, and wherein the kit comprises one or more reagents for performing the method together with instructions for use, and the one or more immunoglobulin levels are selected from the group consisting of total immunoglobulin isotypes and levels of total immunoglobulin isotype subclasses.

39. A kit according to claim 37 wherein the one or more reagents are selected from antibodies, buffers and control reagents.

40. A kit when used in a method of diagnosing or evaluating susceptibility to a cardiovascular disorder, wherein the method involves evaluating a T-cell response in a subject wherein an unregulated TL2 T-cell response and/or suppressed the T-cell response is indicative of susceptibility to, or the presence of, the disorder and the kit comprises one or more reagents for performing the method together with instructions for use.
